SEQUENCE LISTING (9 sequences)

SEQ ID NO: 1 - DNA, 43 bp, Artificial Sequence
  Description of Artificial Sequence Synthetic oligonucleotide
ttcagacgtgtgctcttccgatcttttttttttttttttttva

SEQ ID NO: 2 - DNA, 43 bp, Artificial Sequence
  Description of Artificial Sequence Synthetic oligonucleotide
ttcagacgtgtgctcttccgatcttttttttttttttttttvg

SEQ ID NO: 3 - DNA, 43 bp, Artificial Sequence
  Description of Artificial Sequence Synthetic oligonucleotide
ttcagacgtgtgctcttccgatcttttttttttttttttttvc

SEQ ID NO: 4 - DNA, 43 bp, Artificial Sequence
  Description of Artificial Sequence Synthetic oligonucleotide
ttcagacgtgtgctcttccgatcttttttttttttttttttvt

SEQ ID NO: 5 - DNA, 65 bp, Artificial Sequence
  Description of Artificial Sequence Synthetic oligonucleotide
  modified_base (25)..(27): a, c, t, g, unknown or other
  modified_base (31)..(33): a, c, t, g, unknown or other
  modified_base (37)..(39): a, c, t, g, unknown or other
  modified_base (43)..(45): a, c, t, g, unknown or other
ttcagacgtgtgctcttccgatctnnnaaannnaaannnaaannnttttttttttttttttttva

SEQ ID NO: 6 - DNA, 65 bp, Artificial Sequence
  Description of Artificial Sequence Synthetic oligonucleotide
  modified_base (25)..(27): a, c, t, g, unknown or other
  modified_base (31)..(33): a, c, t, g, unknown or other
  modified_base (37)..(39): a, c, t, g, unknown or other
  modified_base (43)..(45): a, c, t, g, unknown or other
ttcagacgtgtgctcttccgatctnnnaaannnaaannnaaannnttttttttttttttttttvg

SEQ ID NO: 7 - DNA, 65 bp, Artificial Sequence
  Description of Artificial Sequence Synthetic oligonucleotide
  modified_base (25)..(27): a, c, t, g, unknown or other
  modified_base (31)..(33): a, c, t, g, unknown or other
  modified_base (37)..(39): a, c, t, g, unknown or other
  modified_base (43)..(45): a, c, t, g, unknown or other
ttcagacgtgtgctcttccgatctnnnaaannnaaannnaaannnttttttttttttttttttvc

SEQ ID NO: 8 - DNA, 65 bp, Artificial Sequence
  Description of Artificial Sequence Synthetic oligonucleotide
  modified_base (25)..(27): a, c, t, g, unknown or other
  modified_base (31)..(33): a, c, t, g, unknown or other
  modified_base (37)..(39): a, c, t, g, unknown or other
  modified_base (43)..(45): a, c, t, g, unknown or other
ttcagacgtgtgctcttccgatctnnnaaannnaaannnaaannnttttttttttttttttttvt

SEQ ID NO: 9 - DNA, 40 bp, Artificial Sequence
  Description of Artificial Sequence Synthetic oligonucleotide
  misc_feature (1)..(40): This sequence may encompass 18-40 nucleotides
tttttttttttttttttttttttttttttttttttttttt

(19) United States
(12) Patent Application Publication
(10) Pub. No.: US 2020/0208143 A1
(43) Pub. Date: Jul. 2, 2020

(54) METHODS AND COMPOSITIONS FOR AMPLIFYING SHORT DNA FRAGMENTS

(71) Applicant: Paragon Genomics, Inc., Hayward, CA (US)

(72) Inventors: Zhitong LIU, Foster City, CA (US); Chenyu LI, Hayward, CA (US); Lucie LEE, Union City, CA (US); Yang Lily LIU, Oakland, CA (US); Jeffrey Juehui LIU, Foster City, CA (US)

(21) Appl. No.: 16/712,888

(22) Filed: Dec. 12, 2019

Related U.S. Application Data
(60) Provisional application No. 62/778,829, filed on Dec. 12, 2018.

(51) Int. Cl. C12N 15/10; C12Q 1/6869

(52) U.S. Cl. CPC C12N 15/1093; C12Q 1/6869; C12N 15/1013

(57) ABSTRACT

Methods, compositions and systems to amplify short DNA fragments, such as cfDNA, and to reduce random base errors formed in template-dependent primer extension reactions, and to find variant frequencies of mutations on the DNA fragments. The methods, compositions and systems described herein may include, or include the use of, poly(dA) tailing of short DNA fragments by a terminal deoxynucleotidyl transferase, linearly amplification and a multiplex primer extension reaction.

### CROSS REFERENCE TO RELATED APPLICATIONS

This patent application claims priority to U.S. Provisional Patent Application No. 62/778,829, filed on Dec. 12, 2018, titled “METHODS AND COMPOSITIONS FOR AMPLIFYING SHORT DNA FRAGMENTS VIA AN RNA POLYMERASE PROMOTER,” which is herein incorporated by reference in its entirety.

This patent application may be related to U.S. patent application Ser. No. 15/290,981, filed on Oct. 11, 2016, which claims priority as a continuation-in-part to U.S. patent application Ser. No. 15/041,644, filed on Feb. 11, 2016, now U.S. Pat. No. 9,464,318, and titled “METHODS AND COMPOSITIONS FOR REDUCING NON-SPECIFIC AMPLIFICATION PRODUCTS”, which claims priority to U.S. provisional patent applications: U.S. Provisional Patent Application No. 62/114,788, titled “A METHOD FOR ELIMINATING NONSPECIFIC AMPLIFICATION PRODUCTS IN MULTIPLEX PCR” and filed on Feb. 11, 2015; and U.S. Provisional Patent Application No. 62/150,600, titled “METHODS AND COMPOSITIONS FOR REDUCING NON-SPECIFIC AMPLIFICATION PRODUCTS” and filed Apr. 21, 2015. Each of these applications is herein incorporated by reference in its entirety.

This patent application may also be related to international patent application no. PCT/US2018/013143 and U.S. patent application Ser. No. 15/867031, filed on Jan. 10, 2018, which claims priority to U.S. provisional patent applications: U.S. Provisional Patent Application No. 62/444704, titled “METHODS AND COMPOSITIONS FOR REDUCING REDUNDANT MOLECULAR BARCODES CREATED IN PRIMER EXTENSION REACTIONS” and filed on Jan. 10, 2017. Each of these applications is herein incorporated by reference in its entirety.

### SEQUENCE LISTING

The instant application contains a Sequence Listing which has been filed electronically in ASCII format and is hereby incorporated by reference in its entirety. Said ASCII copy, created on Mar. 4, 2020 is named 13982-702_200_SL.txt and is 4,160 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods, compositions, systems and kits described herein relate to the amplification of nucleotide sequences. In particular, the methods, compositions and systems described herein relate to increasing the efficiency of amplifying multiple different DNA fragments and reducing random errors of nucleotide incorporation during amplification. The methods, compositions and systems described herein may include analyzing molecular barcodes and target DNA by high throughput sequencing (next generation sequencing, NGS).

### BACKGROUND

Tumor cells (as well as normal cells) enter into blood and release their DNA in the blood as cell free DNA (cfDNA). cfDNA makes it possible to detect cancer mutations, monitor cancer therapy, and detect gene alterations in prenatal fetus, etc., by drawing a few milliliters of blood and sequencing the cfDNA using high throughput sequencing (e.g., next generation sequencing, NGS). Assaying genetic mutation via blood, termed as liquid biopsy, promises great benefit to the mankind. Detecting mutations in cfDNA is the core technology in liquid biopsy. cfDNA is a mixture of degraded genomic DNA fragments in length from 100 to 200 base pairs. It exists in blood in very low quantities. It therefore demands robust technologies that can amplify cfDNA and detect rare mutations as low as 0.1% allele frequency.

PCR has been a powerful tool to amplify DNA signals. However, because of the length of cfDNA, PCR has difficulty amplify cfDNA with great efficiency. Many cfDNA fragments may not even have one of the primer sites used in PCR. Currently, various technologies of hybridization capture are used to make cfDNA libraries. Hybridization capture usually requires a very long workflow with multiple manipulations of DNA, and also requires a large amount of input cfDNA. These characteristics of hybridization capture limit its use in cfDNA.

Detection of rare mutations by using NGS typically includes enormous amounts of background noises, or random base changes, simultaneously. These random errors arise from the process of library construction, or even the sequencing procedure itself. It is critical to remove these random noises. Unique Molecular Identifies (UMI) is currently the best method in reducing random errors, though it cannot completely eliminate random errors.

Methods of adding unique UMIs onto target DNA by using multiplex PCR have been previously described (e.g., U.S. Pat. No. 10,100,358). This method helped identify the cause of the low efficiency involved in amplifying short DNA fragments, and the origin of the random errors. The methods, compositions and systems (including kits) described herein may address many of the problems described above.

### SUMMARY OF THE DISCLOSURE

In general, described herein are methods, compositions and systems for amplifying short DNA fragments from an RNA polymerase promoter. These methods, compositions and systems may also include a template-dependent multiplex primer extension reaction. These methods, compositions and systems may be useful in any reactions in which a plurality of primers may be used. For example, the methods, compositions and systems described herein may be particularly well suited for use with high throughput sequencing (next generation sequencing, NGS).

Described herein are methods, compositions and systems (e.g., kits, etc.) that use terminal deoxynucleotidyl transferase (also called terminal transferase, TDT) to add a poly(dA) tail onto each single-stranded DNA fragment. These single-stranded DNA fragments are then amplified linearly from an adapter-oligo(dT) primer. Because the offspring products are derived from the identical template, and the errors occurred during DNA synthesis are randomly distributed, this method allows for elimination of random errors through the use of UMIs. The linear amplification products are further amplified for downstream applications.

For example, described herein are methods, compositions and systems for amplifying short DNA fragments and reducing random errors produced in a template-dependent multiplex primer extension reaction. These methods and apparatuses (e.g., systems, kits, etc.) for performing them may include one or more enzymes to adding a poly(dA) tail onto single-stranded DNA fragments, to DNA synthesize in a linear amplification reaction, and amplification of a plurality of targets in a template-dependent multiplex primer extension reaction. The simplicity of the method enables high efficiency and detection of rare mutations from low amount of DNA samples.

It is critical to minimize purification steps to avoid DNA loss during the process of amplifying minute amount of DNA fragments. However, if the high concentration of dATP used in TDT reaction is not removed, it causes dNTP imbalance during the downstream polymerase chain reactions, resulting in artificial mutations. The methods described herein teaches a method that eliminates purification step before target DNA is amplified and the imbalance of dNTPs. The workflow of a method as described herein is schematically illustrated in FIG. 1. Double-stranded DNA fragments are denatured and a poly(dA) tail is added by TDT. After inactivation of TDT and removal of dATP, these DNA fragments are amplified linearly by using an adapter-oligo(dT) primer. The amplification products are purified for the first time after the original target DNA fragments are amplified. Then a plurality of targets is amplified by using a plurality of target-specific primers (e.g., >6, >10, >100, >1000, >10,000, etc.), which may additionally contain a region that serves as unique molecular identifier (UMI, also called molecular barcode). The amplification products can be further amplified while sample barcodes and sequencing adapters are added. The final library can then be used in downstream analysis, such as NGS sequencing.

In the methods described above, the amplification of the target is not limited by the length of the DNA fragments, or the requirement of the presence of two primer sites on a short DNA fragment is eliminated. Any targets, short and long, that harbor one primer site, are amplified. This invention thus allows for amplifying and detecting target signals with significantly higher sensitivity from a limited amount of starting material, such as cfDNA. Further, this invention allows for amplifying and detecting structural change of DNA, such as fusion genes. By modeling the efficiencies of amplifying short DNA fragments, we found the efficiencies of the single-primer amplification method are significantly higher than those of PCR method (FIG. 2). For examples, amplifying a 20 bp target on DNA fragments of 160 bp in length, single-primer method has an efficiency of 72%, while PCR has 56%.

Thus, the methods and apparatuses, including compositions and kits, described herein can amplify a plurality of target short DNA fragments. The length of the shortest target DNA fragment can be 24 bp on average. The length of the target DNA fragments can be 30-200, 40-200, 50-200, 60-200, 70-200,80-200, 90-200, 100-200 bp, etc., in length. Or the length of the target DNA fragments can be 30-1000, 40-1000, 50-1000, 60-1000, 70-1000,80-1000, 90-1000, 100-1000 bp, etc., in length. Or the length of the target DNA fragments can be any numbers of combinations as long as the shortest one is equal or longer than the shortest primer, and the longest one is equal or shorter than the length that can be transcribed by RNA polymerase and reverse transcribed by reverse transcriptase.

In general, the target nucleic acids may comprise DNA or RNA, for example, genomic DNA or cDNA, DNA purified from Formalin-fixed, Paraffin-embedded (FFPE) tissue samples (FFPE DNA), cell-free DNA (cfDNA) or circulating tumor DNA (ctDNA).

DNA fragments are denatured in TDT reaction buffer together with 2 mM dATP by heating to 98° C. for 2 min, followed by quick chilling on ice. TDT is then added to synthesize a poly(dA) tail. The remaining dATP is removed by recombinant Shrimp alkaline phosphatase (rSAP). After the removal of dATP, the enzymes (TDT and rSAP) in the reaction are heat-inactivated together. Poly(dA)-tailed single-stranded DNA fragments are thus produced. In the next step, these poly(dA) tailed DNA fragments are linearly amplified for several cycles in a polymerase chain reaction by directly adding a polymerase, dNTP and an adapter-oligo(dT) to the reaction. Once the targets are linearly amplified, they are purified (offsetting the loss of targets during purification) and ready for further target-specific amplification and enrichment.

In general, the reaction may include exposing single-stranded DNA fragments to 5 Units (U) and 50 U of TDT for 20 minutes and 60 minutes (e.g., 20 minutes, 30 minutes, 40 minutes, 60 minutes, etc.) at 37° C. The removal of dATP may be performed at any appropriate conditions (e.g., concentration, treatment time, temperature, etc.). In general, the reaction may include exposing the reaction mix to 1 Units (U) and 10 U of rSAP for 10 to 30 minutes (e.g., 10 minutes, 20 minutes, 30 minutes, etc.) at 37° C. The heat-inactivation of enzymes may be performed for 10 minutes at 95° C.

An adapter-oligo(dT) is used in linear amplification of the poly(dA)-tailed DNA fragments by PCR. The adapter-oligo(dT) contains an adapter sequence at the 5′ portion and a stretch of Ts at 3′ portion. The adapter serves as a primer site for the downstream amplifications, as well as a sequencing primer site for sequencing applications. The length of the poly(T) is 18 to 40 nucleotides (SEQ ID NO: 9). There may be an anchored 3′ end, as shown in FIGS. 3A and 3B. There may not be a UMI between the adapter and poly(T) (FIG. 3A showing SEQ ID NO: 1 to SEQ ID NO: 4), or there may be a UMI between the adapter and poly(T) (FIG. 3B, showing SEQ ID NO: 5 to SEQ ID NO: 8). The UMI may be a short stretch of random nucleotides (e.g. 8 to 16 random nucleotides), or a short stretch of random nucleotides interspersed by several fixed nucleotides (FIG. 3B).

The linear amplification of the DNA fragments may be performed in a primer extension reaction. In general, the reaction may include annealing of the adapter-oligo(dT) primer to the poly(dA)-tailed DNA fragments and DNA synthesis by a thermostable DNA polymerase. The adapter-oligo(dT) primer may be used at 50 nM to 200 nM. The amplification may be carried out for 3 to 10 cycles.

As mentioned, a template-dependent primer extension reaction may be further included to enrich a plurality of specific targets from the above amplified DNA fragments. The template-dependent primer extension reaction may include any method involving a plurality of oligonucleotides as primers. The length of primers may be from 16-100 nucleotides; the length of amplicons may be from 16 bp-100,000 bp.

The target-specific primers may comprise any appropriate plurality of primers or pairs of primers, such as 7 primers or pairs of primers or more (e.g., at least 7 primers or pairs of primers) of target-specific primers, such as 10 primers or pairs of primers or more (e.g., at least 10 primers or pairs of primers) of target-specific primers, between 7 and 100,000 primers or pairs of primers, between 7 and 1000 primers or pairs of primers, between 1,000 to 100,000 primers or pairs of primers, over 100,000 primers or pairs of primers of target-specific primers, etc., between 10 and 100,000 primers or pairs of primers, between 10 and 1000 primers or pairs of primers, etc. Although seven or more primers or pairs of primers are specified and may be preferable, less than seven pairs may be used (e.g., two or more primers or pairs of primers, three or more primers or pairs of primers, four or more primers or pairs of primers, five or more primers or pairs of primers, or six or more primers or pairs of primers, may be used). The target-specific primers may also comprise any appropriate plurality of primers plus any appropriate plurality of pairs of primers, such as 7 primers plus 7 pairs of primers.

The types of primers that may be used may include unmodified oligonucleotides, modified oligonucleotides, peptide nucleic acid (PNA); modified primers may contain one or more than one 5-methyl deoxycytidine and/or 2,6-diaminopurine, dideoxyinosine, dideoxyuridine, and biotin labeled oligonucleotides. One and/or both primers can contain barcodes or other sequences that allow for identification; one and/or both primers can contain adapter sequences.

Equally important to successful amplifying short DNA fragments by a multiplex primer extension reaction is the efficient removal of non-specific amplification products. Any of the methods described herein may be configured to simultaneously or concurrently degrade non-specific amplification products. In any of the methods described herein, the method may also include removing the degraded non-specific amplification products, leaving the substantial proportion of said plurality of target-specific amplification products. For example, any of the methods described herein may be used with any of the methods or apparatuses described U.S. Pat. No. 9,464,318. Any of the methods described herein may include analyzing the target-specific amplification products. Analyzing may include any appropriate method or technique, including but not limited to sequencing, such as NGS sequencing. Amplification may include any appropriate polynucleotide amplification technique, including in particular a multiplex polymerase chain reaction (PCR).

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an example of a workflow to amplify DNA fragments starting from and poly(dA)-tailing of single-strand DNA fragments by terminal deoxynucleotidyl transferase (TDT). The reaction starts with heat-denaturing DNA fragments. A poly(dA) tail is then added onto the 3′ end of the single-stranded DNA fragments by TDT. The remaining dATP is removed by shrimp alkaline phosphatase (SAP). Both TDT and SAP are then heat-inactivated. The DNA fragments are amplified linearly by PCR with an adapter-oligo(dT) primer. The resulting amplification products are then purified, and used in the downstream step to amplify a plurality of targets in a multiplex PCR reaction. The enriched targets can be further amplified in a PCR reaction to add sample index and sequencing adapters.

FIG. 2 is a model of the efficiency of amplifying short DNA fragments by single-primer and PCR method. The lengths of the DNA fragments are normally distributed in a range of ±33% of the peak. The efficiency of is calculated as eff.=1−(length of amplicon)/(length of DNA peak). The length of amplicon is the summation of the length of primer(s) and the minimal length of the insert. The average length of the primer is 25 nucleotides. The minimal length of the insert for both single-primer method and PCR is 20 bp. To amplify a mixture of DNA fragments with 160 bp peak length, the efficiency of single-primer method is 72%, the efficiency of PCR method is 56%.

FIGS. 3A and 3B illustrate an adapter-oligo(dT) primer. FIG. 3A shows a version of the anchored adapter-oligo(dT) primer. FIG. 3B shows a version of the anchored adapter-oligo(dT) primer with unique molecular identifier (UMI). Note that the UMI in this version of adapter-oligo(dT) primer comprise of 3 random nucleotides interspersed by 3 fixed nucleotides FIG. 3A discloses SEQ ID NOS 1-4, respectively, in order of appearance, while FIG. 3B discloses SEQ ID NOS 5-8, respectively, in order of appearance.

FIG. 4 shows a library generated from 10 ng of 100 bp DNA fragments. The amplified single-stranded DNA fragments were further amplified with a panel of 53 target specific primers, followed by a PCR to add a sample index and sequencing adapters.

FIG. 5 illustrates sequencing result of a library of 53 targets amplified from 10 ng of DNA fragments of 100 bp in length. It shows the sequencing reads of each target in Y-axis and GC percentage of each target in X-axis.

### DETAILED DESCRIPTION

In general, described herein are methods, compositions, systems that may be used to amplify or improve amplification of short target-specific amplification products with reduced number of random errors when amplifying multiple different nucleotide regions. Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Green and Sambrook, Molecular Cloning: A Laboratory Manual (4th Edition, Cold Spring Harbor Laboratory Press, 2012); Rio et al, RNA: A Laboratory Manual (1st Edition, Cold Spring Harbor Laboratory Press, 2010), Berger and Kimmel, Methods in Enzymology, Vol. 152, Guide to Molecular Cloning Techniques (Academic Press, 1987); Alberts et al, Molecular Biology of the Cell (6th Edition, W. W. Norton & Company, 2014), Kornberg and Baker, DNA Replication (2nd Edition, W. H. Freeman, 1992); Nelson and Cox, Lehninger Principles of Biochemistry (7th Edition, W. H. Freeman, 2017); Strachan and Read, Human Molecular Genetics (4th Edition Garland Science, 2010).

“Unique Molecular Identifiers” (UMI) refers to a unique of nucleotide sequence or combination thereof used to label other DNA or RNA molecules. They are usually designed as a string of totally random nucleotides (such as NNNNNNN), partially degenerate nucleotides (such as NNNRNYN), or defined nucleotides (when template molecules are limited). They have given other names, including “molecular barcode”, “molecular index”, “unique identifiers” (UID), “single molecular identifiers” (SMI), “primer ID”, “duplex barcodes”, etc. Molecular barcodes can be as long as 3 to 50 nucleotides, or even longer. They are usually synthesized as a part of the primer or adapter, for example, as a stretch of degenerated nucleotides on either 3′ or 5′ end of adapter. That is, the adapter part has designated nucleotide sequence, the molecular barcode part has random sequences. Molecular barcode can be single stranded, for example, as a part in primer; or double stranded, as it is in adapter. Molecular barcodes are usually added onto the targeted molecules by ligation or through primers during PCR or reverse transcription. Molecular barcodes are used in various applications including, but not limited to, RNA sequencing, studies of single cells, and detection of low frequency mutations. The main purposes of using molecular barcodes are deducing a consensus sequence from the sequences of a group of amplified descendant molecules, thereby to detect the quantity of the original target through removing amplification bias, and finding the true nucleotide sequence of the target through removing random errors and even the false targets. Consensus sequence can be deduced from the amplified sequences of either stand of the target DNA molecule, or collectively from both strands. “Collectively” means the amplified sequences from both of the sense and the antisense strand of the target DNA cannot be differentiated and have to be analyzed together; or the sequences from both strands can be differentiated but be treated as undifferentiated and analyzed together. Complementary double stranded molecular barcodes are used to label both strands of the target molecules, allowing deducing a consensus nucleotide sequence from both strand of the target DNA molecules.

“UMI cluster” means a group of molecular barcodes, on their corresponding target molecules, that have identical or closely related nucleotide sequence. The identical or closely related nucleotide sequence of molecular barcodes of a barcode family is also called as “unique molecular barcode”. “Closely related” means any of the molecular barcodes within one specific family may have one, or two, or three, or any number of different nucleotides, or one, or two, or three, or any number of more or less nucleotides.

“Single strand consensus” means using the sequences from either the sense strand or the antisense strand, or from both of the sense and antisense strand non-discriminatorily of a target DNA molecule to deduce a consensus nucleotide sequence, or the consensus nucleotide sequence deduced from the sequences of either the sense strand or the antisense strand, or from both of the sense and antisense strand non-discriminatorily of the target DNA molecule.

“Double strand consensus” means using the sequences from both of the sense strand and the antisense strand of a target DNA molecule to deduce a consensus nucleotide sequence, or using the sequences from both of a group of the sense strands and a group of the antisense strands of the target DNA molecules to deduce a consensus nucleotide sequence; or the consensus nucleotide sequence deduced from the sequences of the sense strand and the antisense strand of the target DNA molecule, or the consensus nucleotide sequence deduced from the sequences of a group of the sense strands and a group of the antisense strands of the target DNA molecules. Double strand consensus involves, but not limited to, the finding of complementary double stranded molecular barcodes that are used to label both strands of the target molecules, or finding the molecular barcode patterns, as described in this invention, that allows differentiating the sense strands and the antisense strands of the target DNA molecules.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “TAQMAN™” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like. The one or more reagents configured for primer extension reaction and exonuclease cleavage described herein may be configured to include components that permit the primer extension and/or exonuclease cleavage to proceed. For example, one or more reagents configured for primer extension reaction and exonuclease cleavage may include buffering agents to maintain pH at a selected level during a reaction, salts, co-factors, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. A stable duplex can include Watson-Crick base pairing and/or non-Watson-Crick base pairing between the strands of the duplex (where base pairing means the forming hydrogen bonds). In certain embodiments, a non-Watson-Crick base pair includes a nucleoside analog, such as deoxyinosine, 2,6-diaminopurine, PNAs, LNA's and the like. In certain embodiments, a non-Watson-Crick base pair includes a “wobble base”, such as deoxyinosine, 8-oxo-dA, 8-oxo-dG and the like, where by “wobble base” is meant a nucleic acid base that can base pair with a first nucleotide base in a complementary nucleic acid strand but that, when employed as a template strand for nucleic acid synthesis, leads to the incorporation of a second, different nucleotide base into the synthesizing strand (wobble bases are described in further detail below). A “mismatch” in a duplex between two oligonucleotides or polynucleotides means that a pair of nucleotides in the duplex fails to undergo Watson-Crick bonding. “Unmatched base pairs” in a duplex between two oligonucleotides or polynucleotides means that these pairs of nucleotides in the duplex fails to undergo Watson-Crick bonding. A “heteroduplex” region in a duplex between two oligonucleotides or polynucleotides means that the nucleotides on the two strands of this region are unmatched base pairs with each other.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Next-generation sequencing” (NGS) as used herein refers to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (Ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; and Zhang et al., “The impact of next-generation sequencing on genomics”, J Genet Genomics, 2011, 38(3):95-109.

“Nucleotide” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (“LNAs”), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few nanoliters, e.g. 2 nl, to a few hundred μl, e.g. 200 μl. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“TAQMAN™”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified.

“Primer” or “target specific primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, biotin-avidin or biotin-streptavidin interactions, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

“Sample” means a quantity of material from a biological, environmental, medical, or patient source in which detection, measurement, or labeling of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “upstream” and “downstream” in describing nucleic acid molecule orientation and/or polymerization are used herein as understood by one of skill in the art. As such, “downstream” generally means proceeding in the 5′ to 3′ direction, i.e., the direction in which a nucleotide polymerase normally extends a sequence, and “upstream” generally means the converse. For example, a first primer that hybridizes “upstream” of a second primer on the same target nucleic acid molecule is located on the 5′ side of the second primer (and thus nucleic acid polymerization from the first primer proceeds towards the second primer).

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The methods provided herein can be used for amplifying a plurality of short DNA fragments and reducing the random base errors. These methods may involve a plurality of DNA primers or oligonucleotides. The methods disclosed herein provide for optimized protocols such that short DNA fragments are amplified and random base errors are eliminated or reduced. Overall, the methods can relate to improved methods of nucleic acid library preparation.

In one aspect, the methods provide for amplifying short DNA fragments through poly(dA)-tailing and linear amplification followed by primer extension reactions. The method may involve providing a nucleic acid sample comprising at least one target nucleic acid. The nucleic acids can be RNA or DNA. The DNA can be genomic DNA, cDNA, cfDNA, ctDNA or any combination thereof. The DNA can be single-stranded or double-stranded. The DNA can be derived from a eukaryotic cell, an archaea cell, a bacterial cell, a mycobacterial cell, a bacteriophage, a DNA virus, or an RNA virus, or converted from RNA. In some cases, the DNA can be derived from a mammal. In some cases, the DNA can be derived from a human. The DNA can be unmodified, or can be modified (e.g., methylated, glycosylated, etc.). The length of the shortest DNA fragment can be 24 bp. The length of the DNA fragments in the plurality can be 30-200 bp, 40-200 bp, 50-200 bp, 60-200 bp, 70-200 bp, 80-200 bp, 90-200 bp, 100-200 bp, etc., in length. Or the length of the DNA fragments in the plurality can be 30-10,000 bp, 40-10,000 bp, 50-10,000 bp, 60-10,000 bp, 70-10,000 bp, 80-10,000 bp, 90-10,000 bp, 100-10,000 bp, etc., in length. Or the length of the DNA fragments in the plurality can be any numbers of combinations as long as the shortest one is equal or longer than the shortest primer, and the longest one is equal or shorter than the length allowed by the RNA and cDNA synthesis.

In one aspect, the poly(dA) tail is added onto target DNA fragments by TDT, and the DNA fragments are amplified linearly by an adapter-oligo(dT) primer. The adapter-oligo(dT) primer may include a UMI between the adapter and oligo(dT), or may not include a UMI (FIG. 3B). The UMI contains 6-40 random nucleotides. The adapter can comprise unmodified bases and/or phosphodiester bonds, or modified bases and/or phosphodiester bonds, unprotected 5′ ends, or protected 5′ ends, 5′ phosphorylated, or 5′ unphosphorylated ends. It may be blunt-ended, or A-tailed, or T-tailed.

In one aspect, a poly(dA) may be added by TDT onto DNA fragments, and an adapter-oligo(dT) primer is used in the linear amplification step. In some other aspects, a poly(dT) may be added by TDT onto DNA fragments, and an adapter-oligo(dA) primer is used in the linear amplification step. A poly(dG) may be added by TDT onto DNA fragments, and an adapter-oligo(dC) primer is used in the linear amplification step. A poly(dC) may be added by TDT onto DNA fragments, and an adapter-oligo(dG) primer is used in the linear amplification step. In the above aspects, the adapter-oligo(dN) primer may or may not contain a UMI. The UMI may contain 6-40 random nucleotides. Primers may further comprise unmodified bases and/or phosphodiester bonds, or modified bases and/or phosphodiester bonds, unprotected 5′ ends, or protected 5′ ends, 5′ phosphorylated, or 5′ unphosphorylated ends. The linear amplification may be 1 to 10 cycle of polymerase chain reaction (PCR).

A plurality of target-specific primers is used in the downstream primer extension reaction. The plurality of target-specific primers selectively enriches a plurality of target nucleic acids. The plurality of target-specific primers can be in primer pairs, or not in pairs, or in a combination of singular primers and primer pairs. The number of the plurality of target-specific primers can be from 7 to over 100,000 primers. In one case, the plurality of target-specific primers comprises at least 7 target-specific primers. In another case, the plurality of target-specific primers comprises from about 7 to about 100 primers. In another case, the plurality of target-specific primers comprises from about 100 to about 1,000 primers. In yet another case, the plurality of target-specific primers comprises from about 1,000 to about 100,000 primers. In a further case, the plurality of target-specific primers comprises over 100,000 primers.

Multiplex PCR reactions as envisioned in this disclosure can be performed by thermostable DNA polymerases commonly used in PCR reactions. Thermostable DNA polymerases can be wild-type, can have 3′→5′, 5′→3′, or both 3′→5′ and 5′→3′ exonuclease activity, or can be a mixture of thermostable polymerases for higher fidelity, or can synthesize long amplicons, or have faster synthesizing rate. An example of a suitable thermostable DNA polymerase can be Taq DNA polymerase. The thermal profile (temperature and time) for the PCR can be optimized, the primer concentration can also be optimized to achieve the best performance. Finally, any additives that can promote optimal amplification of amplicons can be used. These additives include, without limitation, dimethyl sulfoxide, betaine, formamide, Triton X-100, Tween 20, Nonidet P-40, 4-methylmorpholine N-oxide, tetramethylammonium chloride, 7-deaza-2′-deoxyguanosine, L-proline, bovine serum albumin, trehalose, and T4 gene 32 protein.

The methods as disclosed herein can further involve contacting the reaction with a 3′→5′ single-stranded DNA specific exonuclease for cleaving single-stranded DNA regions and the primers. As used herein, the term “contacting” equates with introducing such enzyme to a pre-existing mixture as described herein. The methods of the present disclosure can use a variety of single-stranded DNA specific exonucleases that can recognize and cleave single-stranded DNA regions in 3′→5′ direction. The plural form will be used herein to refer to enzymes that bind to and cleave aberrant DNA structures. The single-stranded DNA regions include, without limitation, branched DNAs, Y-structures, heteroduplex loops, single stranded overhangs, mismatches, and other kinds of non-perfectly-matched DNAs. In some examples, the single-stranded DNA specific nuclease can reduce the amount of single-stranded DNA regions in the amplification reaction without reducing the amount of target-specific amplification products that do not contain single-stranded DNA regions. In other examples, both single-stranded DNA regions and target-specific amplification products can be reduced. In some cases, the reaction can be substantially free of single-stranded DNA regions. Substantially free of single-stranded DNA regions can mean that the amount of single-stranded DNA regions in the amplification reaction have been reduced by greater than 50%, 60%, 70%, 80%, 85%, 90%, 91%, 92%, 93%, 94%, 95%, 96%, 97%, 98%, 99%, up to 100%.

Examples of 3′→5′ single-stranded DNA specific exonucleases that can be utilized to cleave single-stranded DNA regions in the methods provided herein include, without limitation, exonuclease T, exonuclease I. It should be understood that essentially any 3′→5′ single-stranded DNA specific exonuclease or its mutant that can perform the methods of the disclosure as described herein is envisioned.

In some cases, the methods can involve inactivating the 3′→5′ single-stranded DNA specific exonuclease in the reaction by incubating the reaction at 72° C. for 30 min. In other cases, the 3′→5′ single-stranded DNA specific exonuclease in the reaction is inactivated at 65-72° C. for 10-40 min.

In some cases, the methods as disclosed herein involves the purification of DNA before multiplex PCR. An example method of DNA purification involves DNA purification column, or precipitation by adding one tenth volume of sodium acetate and two-fold volume of pure ethanol. Another example method of DNA purification involves absorption of DNA onto magnetic or paramagnetic micro-beads and elution afterwards.

In some cases, the amplification products described herein can be used to prepare libraries for next-generation sequencing. The common sequences in the primer pairs are identical to part of adapters useful for next-generation sequencing applications. The adapters can be sequencing adapters useful on a next-generation sequencing platform (e.g., Illumina TruSeq adapters). For example, the methods of the invention are useful for next-generation sequencing by the methods commercialized by Illumina, as described in U.S. Pat. No. 5,750,341 (Macevicz); U.S. Pat. No. 6,306,597 (Macevicz); and U.S. Pat. No. 5,969,119 (Macevicz).

Particular reference will now be made to specific aspects and figures of the disclosure. Such aspects are provided by way of example only. Numerous variations, changes, and substitutions will now occur to those skilled in the art without departing from the disclosure.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

An example of the invention described below is to amplify short DNA fragments via poly(dA)-tailing and multiplex primer extension reaction. Short DNA fragments was made by digesting reference DNA NA12878 with Fragmentase from New England Biolab (New England Biolab, catalog number M0348S) according to the suggested method. The lengths of the majority of these DNA fragments are from 100-300 bp. The nucleotide sequence of NA12878 is known. This DNA thus allow us to validate the amplification of short DNA fragments by their nucleotide sequences in next generation sequencing (NGS).

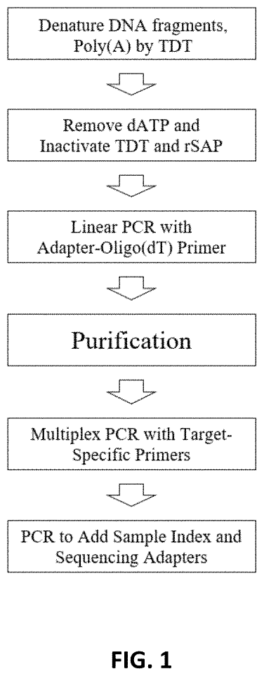

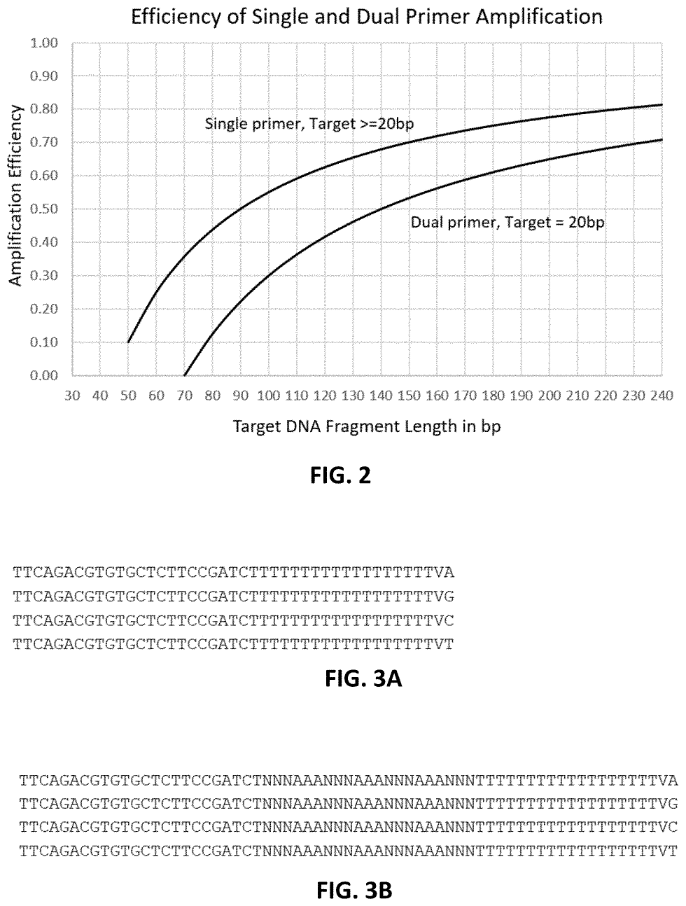

The workflow of the method is depicted in FIG. 1. 10 ng of NA12878 DNA fragments was used in the TDT reaction. The DNA fragments were denatured by heating at 98° C. for 2 minutes in 50 mM TrisHCl, pH 8.3, 50 mM KCl, 5 mM MgCl2, 2 mM dATP. The reaction was then chilled on ice immediately. 10 units of TDT (M0315S, New England Biolabs, Inc.) was added to the reaction and the reaction was incubated for 30 minutes at 37° C. Then 1 units of shrimp alkaline phosphatase (rSAP, M0371S, New England Biolabs, Inc.) was added into the reaction and the reaction was incubated further for 20 minutes at 37° C., followed by heating at 95° C. for 10 minutes to inactivate TDT and SAP. After inactivation of both DTT and SAP, 100 nM of adapter-oligo(dT) (FIG. 3A), 200 μM dNTP and 1 unit of Taq polymerase were added directly into the reaction. A PCR was done for 5 cycles at 34° C. for 1 minute, transition from 34° C. to 68° C. at 0.2° C. per second, 68° C. for 30 seconds and 98° C. for 15 seconds. The above reaction was done in 20 μl.

The DNA was then purified by using 2.5-fold volume of magnetic beads (CleanMag® Magnetic Beads, Paragon Genomics) by following the user guide.

A primer panel containing 53 pairs of primers (CleanPlex® UMI Lung Cancer Panel, Paragon Genomics) was used in the multiplex PCR reaction. Each primer contains a sequencing adapter at 5′ end and a target specific primer at the 3′ end. The adapter is used in a multiplex PCR reaction to enrich specific targets from the above amplified DNA fragments. 100 nM of the panel was used in the multiplex PCR reaction. The multiplex PCR (CleanPlex®, Paragon Genomics) was carried out in 10 μl for 9 cycles at 98° C. for 15 seconds, 60° C. for 5 minutes. After the multiplex PCR, the DNA was purified with 1.6× magnetic beads (CleanMag® Magnetic Beads, Paragon Genomics) by following the user guide.

The purified DNA is subjected to PCR for 10 cycles with primers containing Illumine sequencing adapters. After the PCR, the reaction was further purified by using 1-fold volume of magnetic beads (CleanMag® Magnetic Beads) to generate the library.

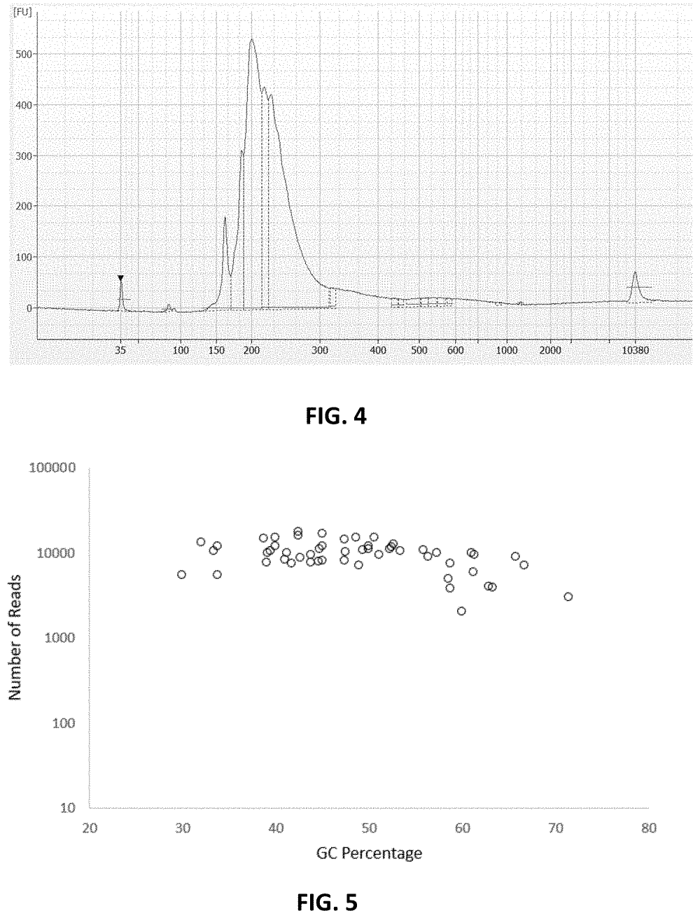

The size, concentration and purity of this library were assayed in a 2100 BioAnalyzer instrument (Agilent Technologies, catalog number G2938B). 1 μl of each library was assayed with a high sensitivity DNA analysis kit (Agilent Technologies, catalog number 5067-4626), according to the methods provided by the supplier. The results are presented in FIG. 4.

To validate the method in amplifying and enriching targets DNA sequences from 10 ng of DNA fragments, the resulting library was sequenced in an Illumina Miseq sequencer. All 53 target sequences were obtained. To demonstrate the efficiency of the method, the distribution of the obtained reads of the targets against their GC percentage is presented in FIG. 5. The uniformity measured by 0.2× mean reads of the obtained targets was 96%.

Any of the methods described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

SEQUENCE LISTING

SEQ ID NO: 1
TTCAGACGTGTGCTCTTCCGATCTTTTTTTTTTTTTTTTTTVA
SEQ ID NO: 2
TTCAGACGTGTGCTCTTCCGATCTTTTTTTTTTTTTTTTTTVG
SEQ ID NO: 3
TTCAGACGTGTGCTCTTCCGATCTTTTTTTTTTTTTTTTTTVC
SEQ ID NO: 4
TTCAGACGTGTGCTCTTCCGATCTTTTTTTTTTTTTTTTTTVT
SEQ ID NO: 5
TTCAGACGTGTGCTCTTCCGATCTNNNAAANNNAAANNNAAAN
NNTTTTTTTTTTTTTTTTTTVA
SEQ ID NO: 6
TTCAGACGTGTGCTCTTCCGATCTNNNAAANNNAAANNNAAAN
NNTTTTTTTTTTTTTTTTTTVG
SEQ ID NO: 7
TTCAGACGTGTGCTCTTCCGATCTNNNAAANNNAAANNNAAAN
NNTTTTTTTTTTTTTTTTTTVC
SEQ ID NO: 8
TTCAGACGTGTGCTCTTCCGATCTNNNAAANNNAAANNNAAAN
NNTTTTTTTTTTTTTTTTTTVT

1. A method of amplifying targets from a plurality of short DNA fragments by using a multiplex primer extension reaction, the method comprising:
designing a plurality of target specific primers to be used as either 5′ end or 3′ end primer in a multiplex primer extension reaction, wherein the 5′ end of said plurality of target specific primers contain a first adapter sequence;
designing an oligo(dT) primer, wherein said oligo(dT) primer comprises, from 5′ to a 3′ end, a second adapter sequence, a region of unique molecular index (UMI) and a stretch of thymines (Ts);
denaturing the plurality of short DNA fragments by heating to above 95° C. into single-stranded DNA fragments, followed by synthesizing a stretch of adenines (As) from the 3′ end of the single stranded DNA fragments by using terminal deoxynucleotidyl transferase and dATP;
synthesizing the complemental strands of the above short DNA fragments by using said oligo(dT) primer;
amplifying a plurality of targets by using the plurality of target specific primers and the second adapter sequence in a multiplex primer extension reaction.

2. The method of claim 1, wherein the plurality of short DNA fragments comprises DNA or cDNA made from RNA.

3. The method of claim 1, wherein the plurality of short DNA fragments is fragmented genomic DNA, fragmented cDNA, fragmented DNA purified from Formalin-fixed, Paraffin-embedded (FFPE) tissue samples (FFPE DNA), cell-free DNA (cfDNA) or circulating tumor DNA (ctDNA).

4. The method of claim 1, wherein the multiplex primer extension reaction comprises multiplex polymerase chain reaction.

5. The method of claim 1, wherein the plurality of target specific primers includes a target-specific region that is complimentary to either sense or anti-sense strand of the plurality of target nucleic acids.

6. The method of claim 1, wherein the plurality of target specific primers comprises either forward primers or reverse primers, or both forward primers and reverse primers.

7. The method of claim 1, wherein the plurality of target specific primers includes a plurality of pairs of primers, wherein each primer pair comprises a forward primer and a reverse primer.

8. The method of claim 1, wherein each primer of the plurality of target specific primers includes a target-specific region that is from 8-50 nucleotides.

9. The method of claim 1, wherein said plurality of target specific primers comprise between 7 target-specific primers and 1,000,000 target-specific primers.

10. The method of claim 1, wherein said plurality of pairs of primers comprise between 7 pairs of target-specific primers and 1,000,000 pairs of target-specific primers.

11. The method of claim 1, wherein each primer of the plurality of target specific primers includes a target-specific region comprising unmodified oligonucleotides.

12. The method of claim 1, wherein each primer of the plurality of target specific primers includes a target-specific region comprising modified oligonucleotides with chemical modifications of nucleotides.

13. The method of claim 1, wherein the first adapter sequence, the second adapter sequence or both the first and second adapter sequences comprises a region of nucleotide sequence used for further amplification and for high-throughput sequencing.

14. The method of claim 1, wherein the unique molecular index comprises 6-40 random nucleotides.

15. The method of claim 14, wherein the random nucleotides are interspersed by a stretch of fixed nucleotides.

16. The method of claim 1, wherein the stretch of Ts in the oligo(dT) primer comprises from 18 to 40 Ts (SEQ ID NO: 9).

17. The method of claim 1, wherein terminal deoxynucleotidyl transferase adds a stretch of As, or a stretch of Ts, or a stretch of guanines (Gs), or a stretch of cytosines (Cs), or a stretch of uracils (Us), to the 3′ end of the single stranded DNA fragments.

18. The method of claim 1, wherein synthesizing the stretch of adenines comprises incubating 1-20 units of terminal deoxynucleotidyl transferase with 0.2-6 mM of a deoxynucleotide and single-stranded DNA fragments at 37° C. for 1-40 minutes.

19. The method of claim 1, further comprising removing the remaining deoxynucleotide after the treatment of terminal deoxynucleotidyl transferase by alkaline phosphatase.

20. The method of claim 19, wherein the alkaline phosphatase is selected from: calf intestinal alkaline phosphatase, Antarctic phosphatase, shrimp alkaline phosphatase;
further comprising adding 1-10 units of alkaline phosphatase to the reaction and incubating at 37° C. for 10-40 minutes.

21. The method of claim 19, further comprising heat inactivating the terminal deoxynucleotidyl transferase and alkaline phosphatase before synthesizing the complemental strands.

22. The method of claim 1, wherein the complement strands of DNA are synthesized from the annealed oligo(dT) primer by using a DNA polymerase.

23. The method of claim 22, wherein the DNA polymerase is chosen from: E. coli DNA polymerase I, Klenow fragment, Taq polymerase, or other thermostable DNA polymerases.

24. The method of claim 1, wherein the oligo(dT) primer is annealed to DNA and the complement strand of DNA is synthesized by using Taq DNA polymerase and slowly increasing the incubation temperature from 30 to 68° C.

25. The method of claim 1, wherein the DNA synthesis from oligo(dT) primer is done in 1-10 cycles, wherein each new cycle of DNA synthesis starts with incubation at 98° C. for 15 seconds.

26. The method of claim 1, wherein the synthesized DNA is further purified using magnetic beads or a DNA purification column.

27. The method of claim 1, wherein the multiplex primer extension reaction is a multiplex polymerase chain reaction.

28. The method of claim 27, further comprising amplifying the products of the multiplex polymerase chain reaction with a pair of primers that are complimentary to the adapter sequences by polymerase chain reaction.

29. The method of claim 28, further comprising analyzing the amplification products by high-throughput sequencing.
